(19) United States
(12) Patent Application Publication
(10) Pub. No.: US 2024/0366422 A1
(43) Pub. Date: Nov. 7, 2024

(54) Targeted Temperature Management Systems, Pads, and Methods Thereof

(71) Applicant: Medivance Incorporated, Louisville, CO (US)

(72) Inventors: Gabriel A. Johnston, Broomfield, CO (US); Michael R. Hoglund, Windsor, CO (US); Madeline Stich, Thornton, CO (US); James David Hughett, Sr., Monroe, GA (US); Sean E. Walker, Platteville, CO (US); Rebecca D. Salisbury, Lafayette, CO (US)

(73) Assignee: Medivance Incorporated, Louisville, CO (US)

(21) Appl. No.: 18/289,341

(22) PCT Filed: Apr. 29, 2022
(86) PCT No.: PCT/US22/26999

§ 371 Date: Nov. 2, 2023

Related U.S. Application Data
(60) Provisional application No. 63/183,506, filed on May 3, 2021.
(60) Provisional application No. 63/185,016, filed on May 6, 2021.

(51) Int. Cl. A61F 7/02

(52) U.S. Cl. CPC A61F 7/02; A61F 2007/0219; A61F 2007/0246; A61F 2007/0274

(57) ABSTRACT

Disclosed are systems, pads, and methods for targeted temperature management. For example, a pad can include a multilayered pad body, a pad inlet connector, and a pad outlet connector. The pad body can include a conduit layer and a patient-interfacing layer over the conduit layer. The conduit layer can include one or more conduits configured to convey a temperature-controlled fluid as a supply fluid from a hydraulic system and a return fluid back to the hydraulic system. The patient-interfacing layer can be configured for placement on a portion of a patient's body. The patient-interfacing layer can have a patient-facing side including a plurality of wells configured to hold a thermally conductive medium. The pad inlet connector can include a pad inlet configured for charging the conduit layer with the supply fluid. The pad outlet connector can include a pad outlet configured for discharging the return fluid from the conduit layer.

### PRIORITY

This application claims the benefit of priority to U.S. Provisional Application No. 63/183,506, filed May 3, 2021, and to U.S. Provisional Application No. 63/185,016, filed May 6, 2021, each of which is incorporated by reference in its entirety into this application.

### BACKGROUND

Targeted temperature management (“TTM”) is a treatment for maintaining therapeutic body temperatures (e.g., hypothermia, hyperthermia, etc.) in patients to improve their outcomes in different medical situations. Current systems for TTM generally use adhesive pads placed on different portions of the patient's bodies to circulate temperature-controlled fluid (e.g., cooled fluid or warmed fluid) about the patients for inducing or maintaining therapeutic body temperatures. Being that the adhesive pads are adhered onto the patient's bodies in order to maintain sufficient contact between the adhesive pads and the patient's bodies for the TTM, improvements to such pads continues to be an active area of research and development.

Disclosed herein are TTM systems, pads, and methods thereof.

### SUMMARY

Disclosed herein is a pad for TTM. The pad includes, in some embodiments, a multilayered pad body, a pad inlet connector, and a pad outlet connector. The multilayered pad body includes a conduit layer and a patient-interfacing layer over the conduit layer. The conduit layer includes one or more conduits configured to convey a temperature-controlled fluid as a supply fluid from a hydraulic system. The one-or-more conduits are also configured to convey a return fluid back to the hydraulic system. The patient-interfacing layer is configured for placement on a portion of a patient's body. The patient-interfacing layer has a patient-facing side including a plurality of wells configured to hold a thermally conductive medium. The pad inlet connector includes a pad inlet configured for charging the conduit layer with the supply fluid. The pad outlet connector includes a pad outlet configured for discharging the return fluid from the conduit layer.

In some embodiments, the wells are square-packed rectangular wells.

In some embodiments, the wells are hexagonally packed circular wells.

In some embodiments, the wells are hexagonally packed hexagonal wells.

In some embodiments, the wells are interconnected.

In some embodiments, the pad includes the thermally conductive medium in the wells.

In some embodiments, the thermally conductive medium includes a polymer, a gel, or a fatty substance.

In some embodiments, the thermally conductive medium includes a solid-particle additive. The solid-particle additive includes metal flakes, metal-oxide particles, salt particles, or carbon particles.

In some embodiments, the thermally conductive medium is a paste of the fatty substance including the solid-particle additive.

In some embodiments, the pad body further includes a film over the patient-interfacing layer individually sealing each well of the plurality of wells. The film is configured for cutting by an instrument, bursting by applied pressure, or dissolving upon contact with skin or a reagent to expose the thermally conductive medium in the wells.

In some embodiments, the pad further includes a release liner over the patient-interfacing layer. The release liner is configured to expose the thermally conductive medium in the wells when the release liner is removed.

In some embodiments, the pad further includes a release liner over the patient-interfacing layer. The release liner is configured to expose the wells when the release liner is removed for subsequent addition of the thermally conductive medium to the wells.

In some embodiments, the pad body further includes an impermeable film between the patient-interfacing layer and the conduit layer. The impermeable film is configured to retain the temperature-controlled fluid in the conduit layer.

In some embodiments, the conduit layer includes a plurality of protrusions extending from the conduit layer toward the impermeable film. The protrusions are configured to promote even flow of the temperature-controlled fluid.

In some embodiments, the pad further includes a secondary fluid delivery line (“FDL”). The secondary FDL is configured to convey the supply fluid from the hydraulic system as well as convey the return fluid back to the hydraulic system. The secondary FDL is split at a pad-connecting end of the secondary FDL. The pad-connecting end of the secondary FDL includes a pair of secondary FDL connectors including a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

Also disclosed is a pad package for TTM. The pad package includes, in some embodiments, one or more pads for TTM, an applicator, and packaging around the one-or-more pads and the applicator. Each pad of the one-or-more pads includes a multilayered pad body. The pad body includes a conduit layer and a patient-interfacing layer over the conduit layer. The conduit layer including one or more conduits configured to convey a temperature-controlled fluid as a supply fluid from a hydraulic system. The one-or-more conduits are also configured to convey a return fluid back to the hydraulic system. The patient-interfacing layer is configured for placement on a portion of a patient's body. The patient-interfacing layer has a patient-facing side including a plurality of wells configured to hold a thermally conductive medium. The applicator includes the thermally conductive medium for addition of the thermally conductive medium to the wells.

In some embodiments, the wells are square-packed rectangular wells, hexagonally packed circular wells, or hexagonally packed hexagonal wells.

In some embodiments, the wells are interconnected.

In some embodiments, the thermally conductive medium includes a polymer, a gel, or a fatty substance.

In some embodiments, the thermally conductive medium includes a solid-particle additive. The solid-particle additive includes metal flakes, metal-oxide particles, salt particles, or carbon particles.

In some embodiments, each pad of the one-or-more pads further includes a release liner over the patient-interfacing layer. The release liner is configured to expose the wells when the release liner is removed for subsequent addition of the thermally conductive medium to the wells.

In some embodiments, the pad body further includes an impermeable film between the patient-interfacing layer and the conduit layer. The impermeable film is configured to retain the temperature-controlled fluid in the conduit layer.

In some embodiments, each pad of the one-or-more pads further includes a pad inlet connector and a pad outlet connector. The pad inlet connector includes a pad inlet configured for charging the conduit layer with the supply fluid. The pad outlet connector includes a pad outlet configured for discharging the return fluid from the conduit layer.

In some embodiments, the pad package further includes one or more secondary FDLs corresponding in number to the one-or-more pads. Each secondary FDL of the one-or-more secondary FDLs is configured to convey the supply fluid from the hydraulic system. Each secondary FDL is also configured to convey the return fluid back to the hydraulic system. Each secondary FDL of the one-or-more secondary FDLs is split at a pad-connecting end of the secondary FDL. The pad-connecting end of the secondary FDL includes a pair of secondary FDL connectors including a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

Also disclosed herein is another pad for TTM. The pad includes, in some embodiments, a two-piece pad body, a pad inlet connector, and a pad outlet connector. A first piece of the pad body includes an impermeable film over a conduit layer. The conduit layer includes one or more conduits configured to convey a temperature-controlled fluid as a supply fluid from a hydraulic system. The one-or-more conduits are also configured to convey a return fluid back to the hydraulic system. The impermeable film is configured to retain the temperature-controlled fluid in the conduit layer. A second double-sided piece of the pad body includes a patient-interfacing side and a pad-interfacing side. The second piece of the pad body is configured for placement on a portion of a patient's body. The first piece of the pad body is configured for placement over the second piece of the pad body. The pad inlet connector includes a pad inlet configured for charging the conduit layer with the supply fluid. The pad outlet connector includes a pad outlet configured for discharging the return fluid from the conduit layer.

In some embodiments, the pad-interfacing side of the second piece of the pad body includes an adhesive thereon. The pad-interfacing side is thusly configured to adhere the first and second pieces of the pad body together when the first piece of the pad body is placed over the second piece of the pad body.

In some embodiments, the impermeable film includes an adhesive thereon. The impermeable film is thusly configured to adhere the first and second pieces of the pad body together when the first piece of the pad body is placed over the second piece of the pad body.

In some embodiments, the patient-interfacing side of the second piece of the pad body includes a plurality of wells configured to hold a thermally conductive medium.

In some embodiments, the wells are square-packed rectangular wells, hexagonally packed circular wells, or hexagonally packed hexagonal wells.

In some embodiments, the wells are interconnected.

In some embodiments, the pad includes the thermally conductive medium in the wells.

In some embodiments, the thermally conductive medium includes a polymer, a gel, or a fatty substance.

In some embodiments, the thermally conductive medium includes a solid-particle additive. The solid-particle additive includes metal flakes, metal-oxide particles, salt particles, or carbon particles.

In some embodiments, the second piece of the pad body further includes a film over the patient-interfacing side individually sealing each well of the plurality of wells. The film is configured for cutting by an instrument, bursting by applied pressure, or dissolving upon contact with skin or a reagent to expose the thermally conductive medium in the wells.

In some embodiments, the pad further includes a release liner over the patient-interfacing side of the second piece of the pad body. The release liner is configured to expose the thermally conductive medium in the wells when the release liner is removed.

In some embodiments, the pad further includes a release liner over the patient-interfacing side of the second piece of the pad body. The release liner is configured to expose the wells when the release liner is removed for subsequent addition of the thermally conductive medium to the wells.

In some embodiments, the patient-interfacing side of the second piece of the pad body includes a hydrogel thereon.

In some embodiments, the hydrogel includes a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, or a combination of two or more of the foregoing hydrogels.

In some embodiments, the pad further includes a release liner over the patient-interfacing side of the second piece of the pad body. The release liner is configured to expose the hydrogel when the release liner is removed.

In some embodiments, the conduit layer includes a plurality of protrusions extending from the conduit layer toward the impermeable film. The protrusions are configured to promote even flow of the temperature-controlled fluid.

In some embodiments, the pad further includes a secondary FDL configured to convey the supply fluid from the hydraulic system. The secondary FDL is also configured to convey the return fluid back to the hydraulic system. The secondary FDL is split at a pad-connecting end of the secondary FDL. The pad-connecting end of the secondary FDL includes a pair of secondary FDL connectors including a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

Also disclosed herein is another pad package for TTM. The pad package includes, in some embodiments, one or more pads for TTM, an applicator, and packaging around the one-or-more pads and the applicator. Each pad of the one-or-more pads includes a two-piece pad body. A first piece of the pad body includes an impermeable film over a conduit layer. The conduit layer includes one or more conduits configured to convey a temperature-controlled fluid as a supply fluid from a hydraulic system. The one-or-more conduits are also configured to convey a return fluid back to the hydraulic system. The impermeable film is configured to retain the temperature-controlled fluid in the conduit layer. A second double-sided piece of the pad body includes a patient-interfacing side and a pad-interfacing side. The patient-interfacing side of the second piece of the pad body includes a plurality of wells configured to hold a thermally conductive medium. The second piece of the pad body is configured for placement on a portion of a patient's body. The first piece of the pad body configured for placement over the second piece of the pad body. The applicator includes the thermally conductive medium for addition of the thermally conductive medium to the wells.

In some embodiments, the wells are square-packed rectangular wells, hexagonally packed circular wells, or hexagonally packed hexagonal wells.

In some embodiments, the wells are interconnected.

In some embodiments, the thermally conductive medium includes a polymer, a gel, or a fatty substance.

In some embodiments, the thermally conductive medium includes a solid-particle additive. The solid-particle additive includes metal flakes, metal-oxide particles, salt particles, or carbon particles.

In some embodiments, each pad of the one-or-more pads further includes a release liner over the patient-interfacing side of the second piece of the pad body. The release liner is configured to expose the wells when the release liner is removed for subsequent addition of the thermally conductive medium to the wells.

In some embodiments, the pad-interfacing side of the second piece of the pad body includes an adhesive thereon. The pad-interfacing side of the second piece of the pad body is thusly configured to adhere the first and second pieces of the pad body together when the first piece of the pad body is placed over the second piece of the pad body.

In some embodiments, the impermeable film includes an adhesive thereon. The impermeable film is thusly configured to adhere the first and second pieces of the pad body together when the first piece of the pad body is placed over the second piece of the pad body.

In some embodiments, each pad of the one-or-more pads further includes a pad inlet connector and a pad outlet connector. The pad inlet connector includes a pad inlet configured for charging the conduit layer with the supply fluid. The pad outlet connector includes a pad outlet configured for discharging the return fluid from the conduit layer.

In some embodiments, the pad package further includes one or more secondary FDLs corresponding in number to the one-or-more pads. Each secondary FDL of the one-or-more secondary FDLs is configured to convey the supply fluid from the hydraulic system. Each secondary FDL is also configured to convey the return fluid back to the hydraulic system. Each secondary FDL of the one-or-more secondary FDLs is split at a pad-connecting end of the secondary FDL. The pad-connecting end of the secondary FDL includes a pair of secondary FDL connectors including a secondary FDL outlet connector and a secondary FDL inlet connector. The secondary FDL outlet connector is configured to fluidly connect to the pad inlet connector. The secondary FDL inlet connector is configured to fluidly connect to the pad outlet connector.

Also disclosed herein is a system for TTM. The system includes, in some embodiments, a control module, a primary FDL, and one or more pads selected from paragraphs [0003]-[0016] and [0025]-[0040]. The control module includes a hydraulic system configured to provide a temperature-controlled fluid. The primary FDL is configured to convey the temperature-controlled fluid from the hydraulic system as a supply fluid. The primary FDL is also configured to convey the temperature-controlled fluid back to the hydraulic system as a return fluid. The one-or-more pads are configured for placement on one or more portions of a patient's body, respectively.

In some embodiments, the hydraulic system includes a chiller evaporator, a heater, a hydraulic-system outlet, and a hydraulic-system inlet. The chiller evaporator is configured for fluid cooling. The heater is configured for fluid heating. The chiller evaporator and the heater, together, are configured to provide the temperature-controlled fluid. The hydraulic-system outlet is configured for discharging the supply fluid from the hydraulic system. The hydraulic-system inlet is configured for charging the hydraulic system with the return fluid to continue to produce the temperature-controlled fluid.

In some embodiments, the control module further includes one or more processors, primary memory, and instructions stored in the primary memory. The instructions are configured to instantiate one or more processes for TTM with the control module when executed by the one-or-more processors.

Also disclosed herein is a method of a system for TTM. The method includes, in some embodiments, a pad-placing step, a pad-charging step, and a fluid-circulating step. The pad-placing step includes placing at least a portion of a pad on a patient's body in contact with skin of the patient's body. The portion of the pad in contact with the skin of the patient's body includes a plurality of wells in a patient-facing side of a patient-interfacing layer of a multilayered pad body of the pad. Alternatively, the portion of the pad in contact with the skin of the patient's body includes the plurality of wells in a double-sided second piece of two pieces of the pad. The wells include a thermally conductive medium therein. The pad-charging step includes charging one or more conduits of a conduit layer of the pad with a supply fluid of a temperature-controlled fluid. The conduit layer is either part of the multilayered pad body or part of a first piece of the two pieces of the pad. The supply fluid is provided by a hydraulic system of a control module by way of a combination of fluidly connected FDLs including a secondary FDL and a primary FDL. The fluid-circulating step includes circulating the supply fluid through the conduit layer to cool or warm the patient's body as needed in accordance with TTM.

In some embodiments, the method includes a film-cutting step. The film-cutting step includes cutting a film over the wells with an instrument to expose the thermally conductive medium in any one or more individually sealed wells of the plurality of wells. The film-cutting step is performed before the pad-placing step.

In some embodiments, the method includes a film-bursting step. The film-bursting step includes bursting a film over the wells with applied pressure to expose the thermally conductive medium in any one or more individually sealed wells of the plurality of wells. The film-bursting step is performed before the pad-placing step.

In some embodiments, the method includes a film-dissolving step. The film-dissolving step includes dissolving a film over the wells upon contact with patient skin or a reagent to expose the thermally conductive medium in any one or more individually sealed wells of the plurality of wells. The film-dissolving step is performed before the pad-placing step.

In some embodiments, the method further includes a release liner-removing step. The release liner-removing step includes removing a release liner from the pad to expose the wells including the thermally conductive medium therein. The release liner-removing step is performed before the pad-placing step.

In some embodiments, the method further includes a release liner-removing step and a dispensing step. The release liner-removing step includes removing a release liner from the pad to expose the wells. The dispensing step includes dispensing the thermally conductive medium into the wells. Both the release liner-removing step and the dispensing step are performed before the pad-placing step.

In some embodiments, the method further comprises an FDL-connecting step. The FDL-connecting step includes fluidly connecting a secondary FDL outlet connector at a split pad-connecting end of the secondary FDL to a pad inlet connector. The FDL-connecting step also includes fluidly connecting a secondary FDL inlet connector at the split pad-connecting end of the secondary FDL to a pad outlet connector.

In some embodiments, the fluid-circulating step includes transferring heat between the temperature-controlled fluid and the patient's body by thermal conduction through the conduit layer.

In some embodiments, the fluid-circulating step includes circulating a cool fluid through the conduit layer to bring the patient into hypothermia from normothermia.

In some embodiments, the fluid-circulating step includes circulating a warm fluid through the conduit layer to bring the patient into normothermia from hypothermia.

In some embodiments, the fluid-circulating step includes circulating a warm fluid through the conduit layer to bring the patient into hyperthermia from normothermia.

In some embodiments, the fluid-circulating step includes circulating a cool fluid through the conduit layer to bring the patient into normothermia from hyperthermia.

Also disclosed herein is another method of a system for TTM. The method includes, in some embodiments, a pad-placing step, a pad-charging step, and a fluid-circulating step. The pad-placing step includes placing a double-sided second piece of a two-piece pad on a patient's body in contact with skin of the patient's body. The second piece of the pad includes a hydrogel on a patient-facing side of the second piece. The pad-placing step also includes placing a first piece of the pad over the second piece of the pad. The pad-charging step includes charging one or more conduits of a conduit layer or the first piece of the pad with a supply fluid of a temperature-controlled fluid. The supply fluid is provided by a hydraulic system of a control module by way of a combination of fluidly connected FDLs including a secondary FDL and a primary FDL. The fluid-circulating step includes circulating the supply fluid through the conduit layer to cool or warm the patient's body as needed in accordance with TTM.

In some embodiments, the method further includes a release liner-removing step. The release liner-removing step includes removing a release liner from the patient-facing side of the second piece of the pad to expose the hydrogel. The release liner-removing step is performed before placing the pad-placing step.

In some embodiments, the method further includes another release liner-removing step. The other release liner-removing step includes removing a release liner from a pad-facing side of the second piece of the pad to expose an adhesive configured to adhere the first and second pieces of the pad body together upon placing the first piece of the pad over the second piece of the pad in the pad-placing step.

In some embodiments, the method further comprises an FDL-connecting step. The FDL-connecting step includes fluidly connecting a secondary FDL outlet connector at a split pad-connecting end of the secondary FDL to a pad inlet connector. The FDL-connecting step also includes fluidly connecting a secondary FDL inlet connector at the split pad-connecting end of the secondary FDL to a pad outlet connector.

In some embodiments, the fluid-circulating step includes transferring heat between the temperature-controlled fluid and the patient's body by thermal conduction through the conduit layer.

In some embodiments, the fluid-circulating step includes circulating a cool fluid through the conduit layer to bring the patient into hypothermia from normothermia.

In some embodiments, the fluid-circulating step includes circulating a warm fluid through the conduit layer to bring the patient into normothermia from hypothermia.

In some embodiments, the fluid-circulating step includes circulating a warm fluid through the conduit layer to bring the patient into hyperthermia from normothermia.

In some embodiments, the fluid-circulating step includes circulating a cool fluid through the conduit layer to bring the patient into normothermia from hyperthermia.

These and other features of the concepts provided herein will become more apparent to those of skill in the art in view of the accompanying drawings and following description, which describe particular embodiments of such concepts in greater detail.

### DRAWINGS

FIG. 1 illustrates a system for TTM including a console and one or more pads in accordance with some embodiments.

FIG. 2A illustrates left and right torso pads of the one-or-more pads in accordance with some embodiments.

FIG. 2B illustrates left and right leg pads of the one-or-more pads in accordance with some embodiments.

FIG. 3A illustrates a multilayered pad body of a pad of the one-or-more pads in accordance with some embodiments.

FIG. 3B illustrates a two-piece pad body of a pad of the one-or-more pads in accordance with some embodiments.

FIG. 4A illustrates a plurality of wells in the pad of FIG. 3A or 3B in accordance with some embodiments.

FIG. 4B illustrates another plurality of wells in the pad of FIG. 3A or 3B in accordance with some embodiments.

FIG. 4C illustrates yet another plurality of wells in the pad of FIG. 3A or 3B in accordance with some embodiments.

FIG. 4D illustrates yet another plurality of wells in the pad of FIG. 3A or 3B in accordance with some embodiments.

FIG. 4E illustrates one or more channels in the pad of FIG. 3A or 3B in accordance with some embodiments.

FIG. 5 illustrates a hydraulic system of a control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, TTM is a treatment for maintaining therapeutic body temperatures (e.g., hypothermia, hyperthermia, etc.) in patients to improve their outcomes in different medical situations. Current systems for TTM generally use adhesive pads placed on different portions of the patient's bodies to circulate temperature-controlled fluid (e.g., cooled fluid or warmed fluid) about the patients for inducing or maintaining therapeutic body temperatures. Being that the adhesive pads are adhered onto the patient's bodies in order to maintain sufficient contact between the adhesive pads and the patient's bodies for the TTM, improvements to such pads continues to be an active area of research and development. Disclosed herein are TTM systems, pads, and methods thereof.

### Systems for TTM

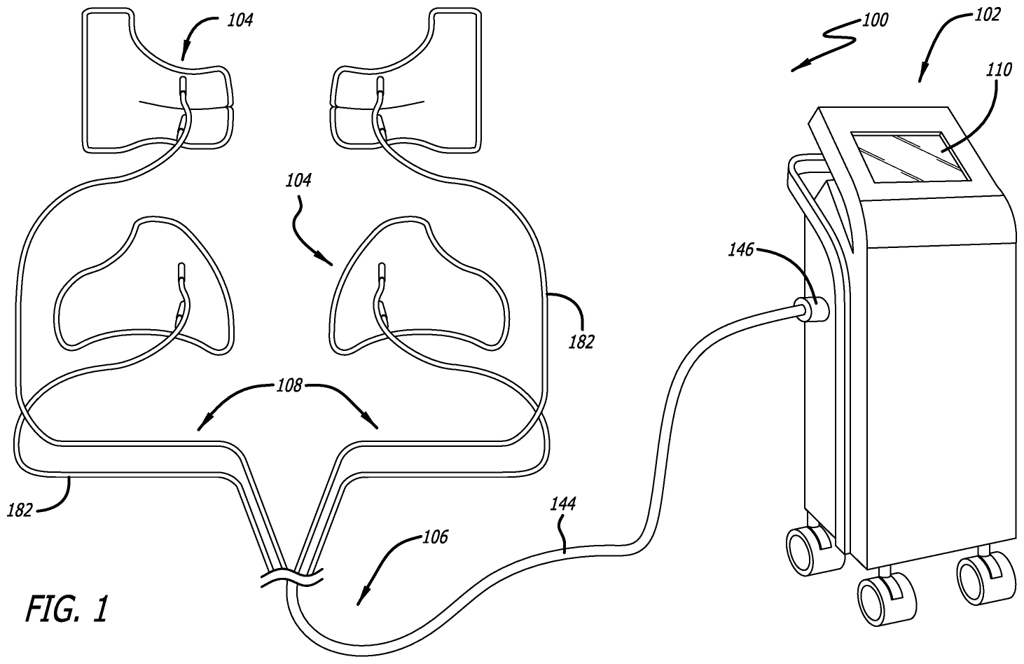

FIG. 1 illustrates a system 100 for TTM including a control module 102 and one or more pads 104 in accordance with some embodiments.

As shown, the system 100 can include the control module 102, the one-or-more pads 104 such as any of those set forth below, a primary FDL 106, and one or more secondary FDLs 108 corresponding in number to the one-or-more pads 104. Description for the control module 102 is set forth immediately below. Description for the one-or-more pads 104 and the one-or-more secondary FDLs 108 is set forth in the following section.

The control module 102 can include a console 110 with an integrated display screen configured as a touchscreen for operating the control module 102. The control module 102 can include one or more processors, primary and secondary memory, and instructions stored in the primary memory. The instructions are configured to instantiate one or more processes for TTM with the control module 102 when executed by the one-or-more processors.

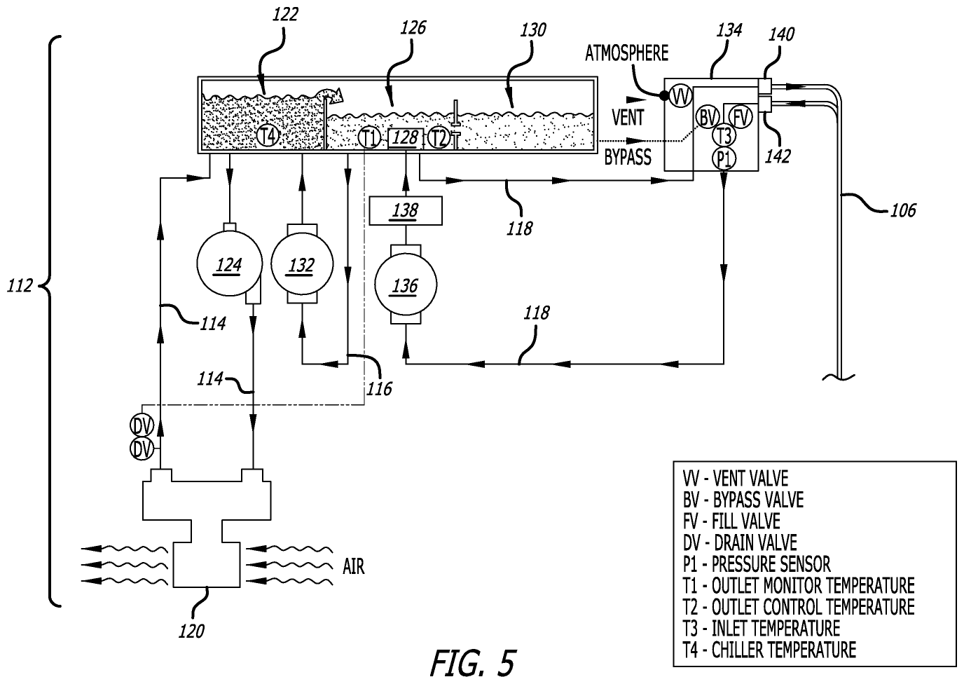

FIG. 5 illustrates a hydraulic system 112 of the control module 102 in accordance with some embodiments.

The control module 102 can also include the hydraulic system 112, which can include a chiller circuit 114, a mixing circuit 116, and a circulating circuit 118 for providing a temperature-controlled fluid.

The chiller circuit 114 can be configured for cooling a fluid (e.g., water, ethylene glycol, a combination of water and ethylene glycol, etc.) to produce a cooled fluid, which cooled fluid, in turn, can be for mixing with the mixed fluid in the mixing tank 126 set forth below to produce a supply fluid for TTM. The chiller circuit 114 can include a chiller evaporator 120 configured for the cooling of the fluid passing therethrough. The fluid for the cooling by the chiller evaporator 120 is provided by a chiller tank 122 using a chiller pump 124 of the chiller circuit 114.

The mixing circuit 116 can be configured for mixing spillover of the cooled fluid from the chiller tank 122 with a mixed fluid in a mixing tank 126 of the mixing circuit 116. The mixing circuit 116 can include a heater 128 in the mixing tank 126 configured for heating the mixed fluid to produce a heated fluid, which can be mixed with the cooled fluid in any ratio to provide a supply tank 130 of the circulating circuit 118 with the supply fluid of a desired temperature for TTM. Indeed, the chiller evaporator 120 and the heater 128, together, are configured to cooperate to provide the temperature-controlled fluid. The mixing circuit 116 can include a mixing pump 132 configured to pump the fluid from the mixing tank 126 into the chiller tank 122 for producing the cooled fluid as well as the spillover of the cooled fluid for the mixing tank 126.

The circulating circuit 118 can be configured for circulating the supply fluid for TTM, which includes circulating the supply fluid provided by a manifold 134 through the one-or-more pads 104 using a circulation pump 136 directly or indirectly governed by a flow meter 138 of the circulating circuit 118. The manifold 134 can include an outlet 140 configured for discharging the supply fluid (e.g., a cooled fluid or a warmed fluid as indicated) from the hydraulic system 112 and an inlet 142 configured for charging the hydraulic system 112 with return fluid from the one-or-more pads 104 to continue to produce the supply fluid.

The primary FDL 106 can include primary tubing 144 configured to convey the supply fluid from the hydraulic system 112 by way of a lumen of the primary tubing 144 when fluidly connected to the hydraulic system 112. Likewise, the primary tubing 144 is configured convey the return fluid back to the hydraulic system 112 by way of the lumen of the primary tubing 144 when fluidly connected to the hydraulic system 112.

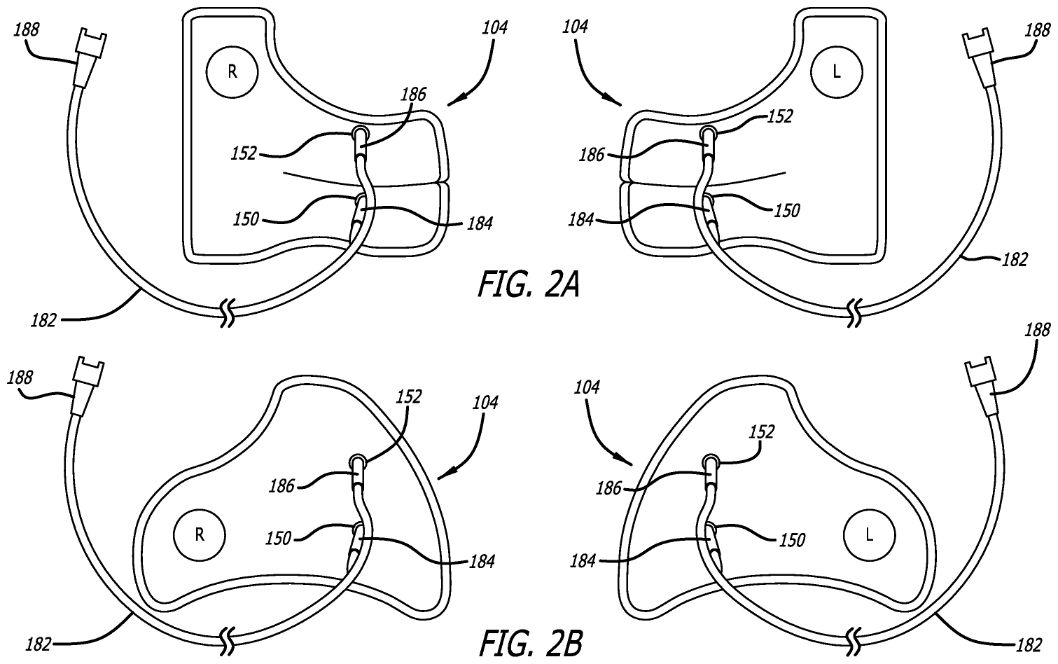

The primary tubing 144 of the primary FDL 106 of FIG. 1 can include a pair of opposing connector ends, wherein each connector end of the pair of connector ends includes a corresponding primary FDL connector. For example, a connector end of the pair of connector ends can include a control module-connecting primary FDL connector 146 configured to fluidly connect to a control-module connector (not shown) of the control module 102. The control-module connector can include both the outlet 140 and the inlet 142 of the hydraulic system 112. Another connector end of the pair of connector ends can include a secondary FDL-connecting primary FDL connector (not shown) configured to fluidly connect to the primary FDL-connecting secondary FDL connector 188 set forth below. (See FIGS. 2A and 2B.) The secondary FDL-connecting primary FDL connector can include both an outlet and an inlet.

### Pads for TTM

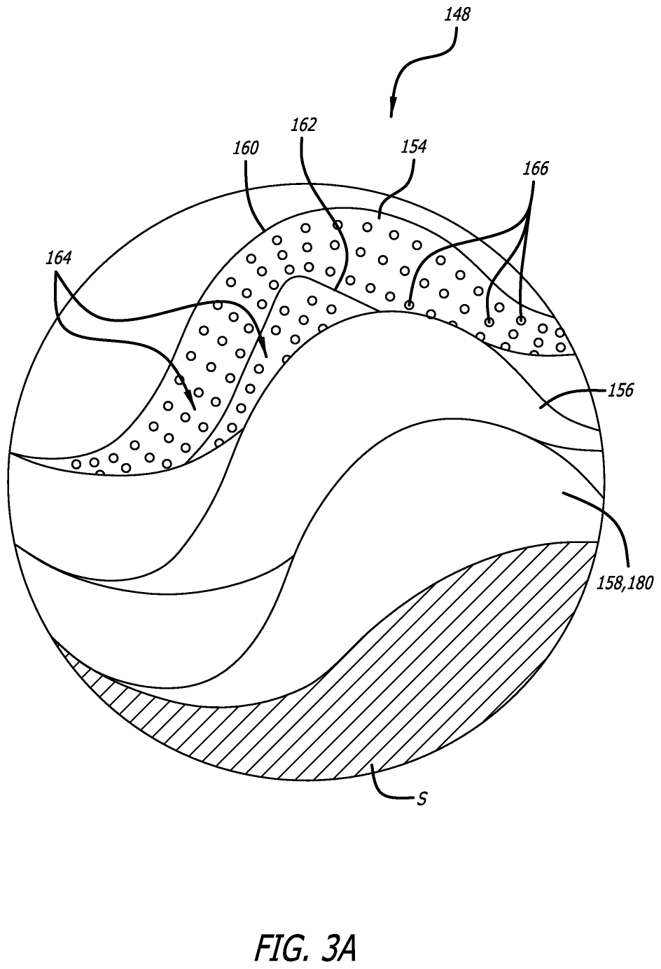

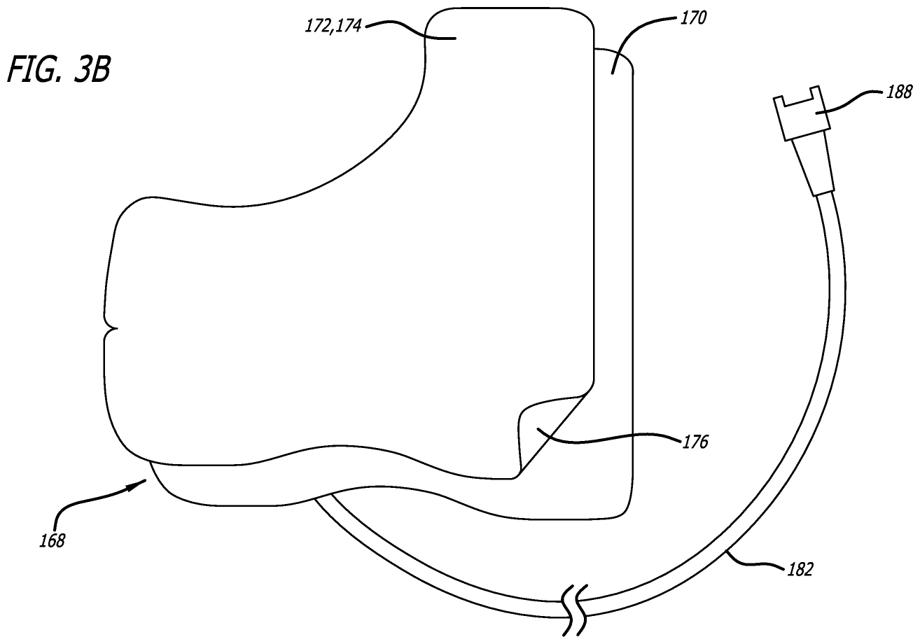

FIGS. 2A and 2B illustrate left and right pads of the one-or-more pads 104 respectively for a torso and legs of a patient in accordance with some embodiments. However, the one-or-more pads 104 can be configured for placement on any one or more portions of a patient's body, respectively, not just the torso or legs. FIG. 3A illustrates a multilayered pad body 148 in accordance with some embodiments of a pad of the one-or-more pads 104, whereas FIG. 3B illustrates a two-piece pad body 168 in accordance with some other embodiments of a pad of the one-or-more pads 104.

Beginning with the embodiment of the pad of the one-or-more pads 104 shown among FIGS. 2A, 2B, and 3A, the pad can include the multilayered pad body 148, a pad inlet connector 150, and a pad outlet connector 152.

The pad body 148 can include a conduit layer 154, an impermeable film 156 over the conduit layer 154, and a patient-interfacing layer 158 over both the conduit layer 154 and the impermeable film 156.

The conduit layer 154 can include a perimetrical wall 160 and one or more inner wall 162s extending from the conduit layer 154 toward the impermeable film 156. Together with the impermeable film 156, the perimetrical wall 160 and the one-or-more inner walls 162 form one or more conduits 164 configured to convey through the conduit layer 154 the temperature-controlled fluid as the supply fluid from the hydraulic system 112 or the return fluid back to the hydraulic system 112.

The conduit layer 154 can include a plurality of protrusions 166 extending from the conduit layer 154 toward the impermeable film 156. The protrusions 166 can be configured to promote even flow of the temperature-controlled fluid as the supply fluid or the return fluid when conveyed through the conduit layer 154.

The conduit layer 154 can be a unitary piece of an opaque polymer (e.g., foam) or a translucent polymer such as an elastomer (e.g., silicone).

The impermeable film 156 can be configured to retain the temperature-controlled fluid as the supply fluid or the return fluid in the conduit layer 154 when the conveyed through the conduit layer 154. In addition, the impermeable film 156 can be configured to allow efficient energy transfer between the conduit layer 154 and the patient-interfacing layer 158.

The patient-interfacing layer 158 can be configured for placement on skin S (see FIG. 3A) of a portion (e.g., torso, leg, etc.) of a patient's body for thermal conduction through the patient-interfacing layer 158.

Adverting to the embodiment of the pad of the one-or-more pads 104 shown among FIGS. 2A, 2B, and 3B, the pad can include a two-piece pad body 168, the pad inlet connector 150, and the pad outlet connector 152.

A first piece 170 of the pad body 168 can include the conduit layer 154 and the impermeable film 156 over the conduit layer 154 like the pad body 148 set forth above. Again, the conduit layer 154 can include the one-or-more conduits 164 configured to convey through the conduit layer 154 the temperature-controlled fluid as the supply fluid from the hydraulic system 112 or the return fluid back to the hydraulic system 112. The impermeable film 156 can be configured to retain the temperature-controlled fluid in the conduit layer 154 as set forth above.

A second, double-sided piece 172 of the pad body 168 can include a patient-interfacing side 174 and a pad-interfacing side 176. The second piece 172 of the pad body 168 can be configured for placement on a portion of a patient's body with the patient-interfacing side 174 of the second piece 172 in contact with skin S of the patient. (See, for example, FIG. 3A.) The first piece 170 of the pad body 168 can be configured for placement over the pad-interfacing side 176 of the second piece 172 of the pad body 168.

Beginning with the pad-interfacing side 176 of the second piece 172 of the pad body 168, the pad-interfacing side 176 can include an adhesive thereon configured to adhere the first and second pieces 170 and 172 of the pad body 168 together when the first piece 170 is placed over the second piece 172. Alternatively, the impermeable film 156 of the first piece 170 of the pad body 168 includes the adhesive thereon to adhere the first and second pieces 170 and 172 of the pad body 168 together when the first piece 170 is placed over the second piece 172. While not shown, the pad-interfacing side 176 of the second piece 172 of the pad body 168 can include a release liner over the adhesive if present on the pad-interfacing side 176 of the second piece 172. Likewise, the impermeable film 156 of the first piece 170 of the pad body 168 can include a release liner over the adhesive if present on the impermeable film 156 of the first piece 170. Such a release liner, which maintains an integrity of the pad-interfacing side 176 of the second piece 172 of the pad body 168 or the impermeable film 156 of the second piece 172 of the pad body 168, can be configured to expose the adhesive when the release liner is removed from the pad.

Adverting to the patient-interfacing side 174 of the second piece 172 of the pad body 168, the patient-interfacing side 174 can include a hydrogel thereon. The hydrogel can be configured to conformably adhere to a portion of a patient's body for optimum thermal conduction but without irritation. Such a hydrogel can be selected from a poly(ethylene glycol) hydrogel, an alginate-based hydrogel, a chitosan-based hydrogel, a collagen-based hydrogel, a dextran-based hydrogel, a hyaluronan-based hydrogel, a xanthan-based hydrogel, a konjac-based hydrogel, a gelatin-based hydrogel, and a combination of two or more of the foregoing hydrogels. While not shown, the patient-interfacing side 174 of the second piece 172 of the pad body 168 can include a release liner over the hydrogel on the patient-interfacing side 174. Such a release liner, which maintains an integrity of the patient-interfacing side 174 of the second piece 172 of the pad body 168, can be configured to expose the hydrogel when the release liner is removed from the pad.

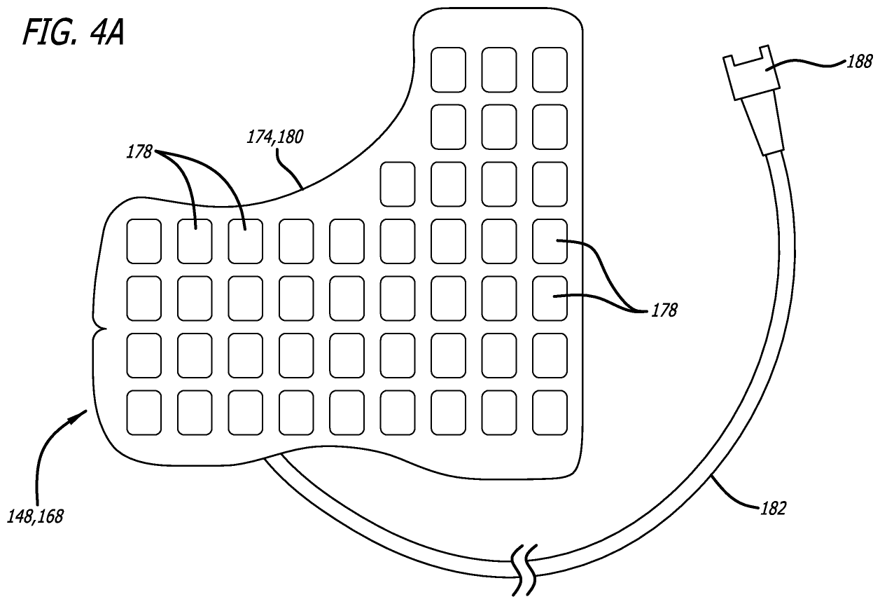

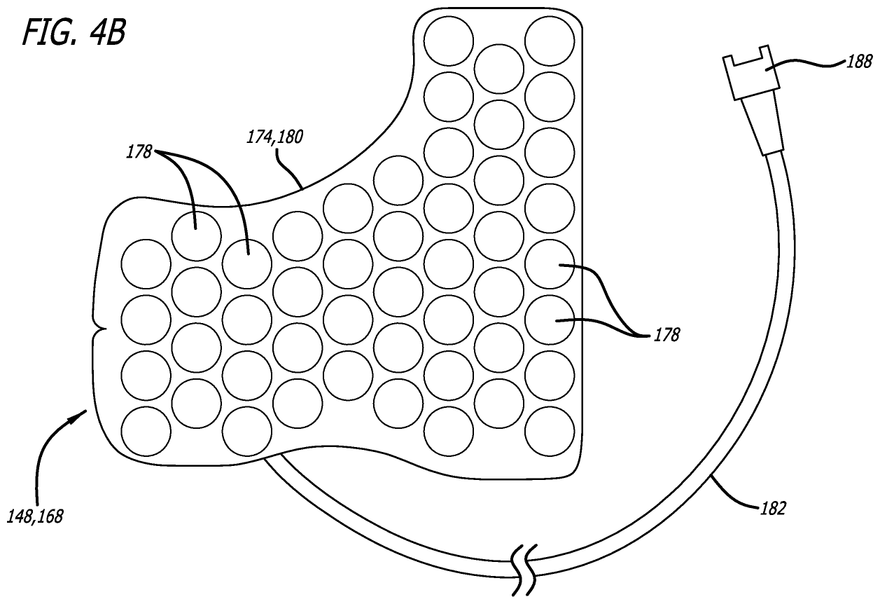

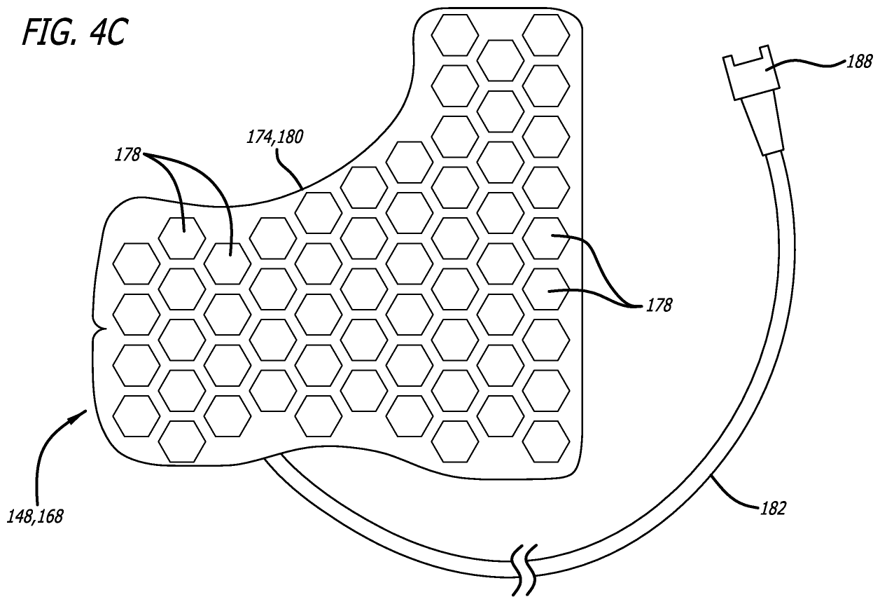

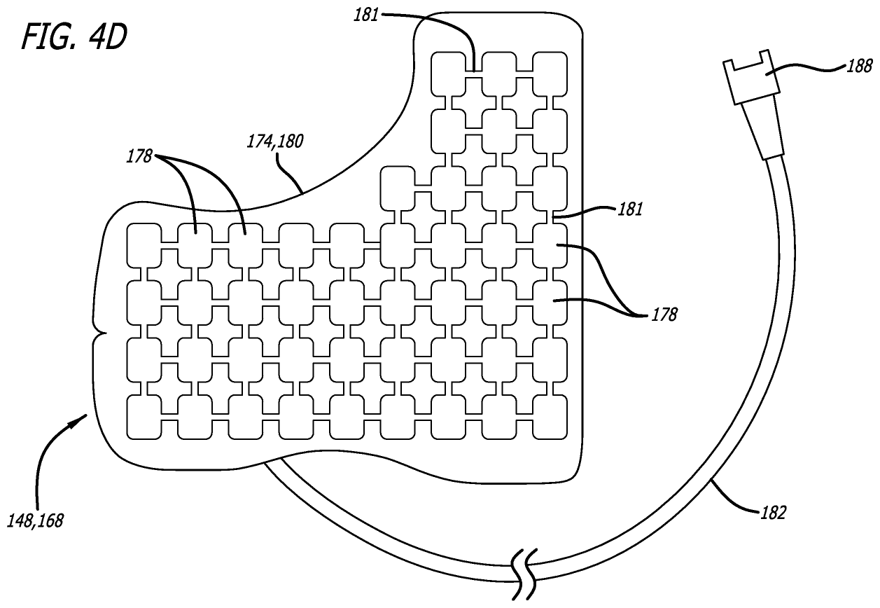

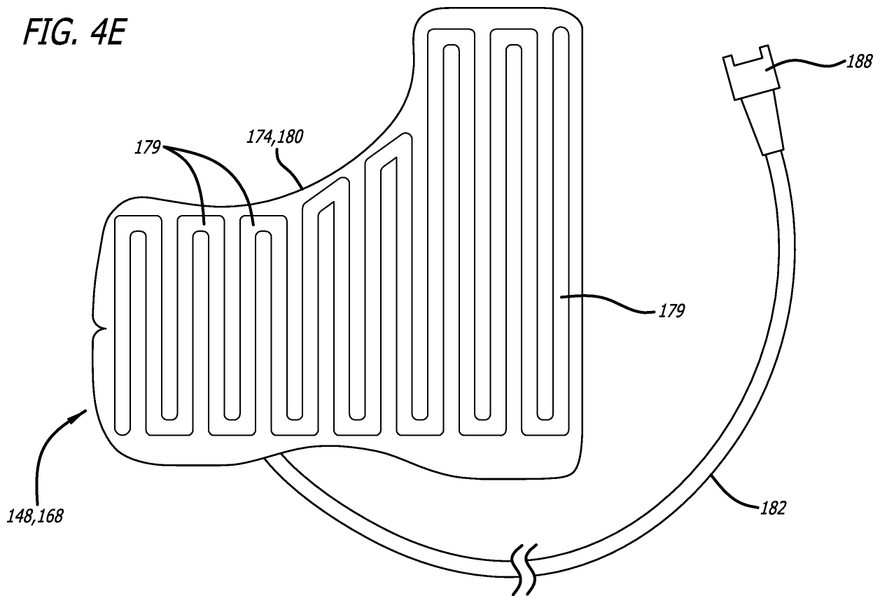

FIGS. 4A-4D illustrate a plurality of wells 178 in the pad of FIG. 3A or 3B in accordance with some embodiments. FIG. 4E illustrates one or more channels 179 in the pad of FIG. 3A or 3B in accordance with some embodiments.

Adverting back to the embodiment of the pad of the one-or-more pads 104 shown among FIGS. 2A, 2B, and 3A, as well as another embodiment of the pad of the one-or-more pads 104 shown among FIGS. 2A, 2B, and 3B, each patient-interfacing side of a patient-interfacing side 180 of the patient-interfacing layer 158 of the pad body 148 and the patient-interfacing side 174 of the second piece 172 of the pad body 168 can include the wells 178 or the one-or-more channels 179 therein. The wells 178 can be square-packed rectangular wells as shown in FIGS. 4A and 4D, hexagonally packed circular wells as shown in FIG. 4B, hexagonally packed hexagonal wells as shown in FIG. 4C, or the like. Indeed, the wells 178 can have any shape or combination of shapes in any arrangement or combination of arrangements sufficient for thermal conduction therethrough when combined with the thermally conductive medium set forth below. In addition, the wells 178 can be interconnected with connecting channels 181 as shown for the square-packed rectangular wells of FIG. 4E. Interconnection of the wells 178 by the connecting channels 181 can facilitate initial application and distribution of the thermally conductive medium from, for example, the applicator set forth below. Interconnection of the wells 178 by the connecting channels 181 can also facilitate continued distribution of the thermally conductive medium upon, for example, patient movement. While not shown, the one-or-more channels 179 can include the connecting channels 181 as well. Like those for the wells 178, the connecting channels 181 can also facilitate application and distribution of the thermally conductive medium in the one-or-more channels 179.

The wells 178 and the one-or-more channels 179 are configured to hold therein a thermally conductive medium, which thermally conductive medium can be included in the wells 178 or the one-or-more channels 179 of each pad of the one-or more pads 104 as packaged. Alternatively, the thermally conductive medium can be separately provided in a same or different package as the one-or-more pads 104. The thermally conductive medium can include, but is not limited to a polymer, a gel, or a fatty substance. For example, the polymer can be a polysiloxane such as silicone; the gel can be a networked polymer such as poly(acrylic acid), poly(vinyl alcohol), poly(vinylpyrrolidone), poly(ethylene glycol), polyacrylamide, or the like including a swelling agent such as water, thereby providing a hydrogel; and the fatty substance can be petroleum jelly or a lipid such as an ester of one or more fatty acids. In addition, the thermally conductive medium can include a solid-particle additive. The solid-particle additive can include metal flakes, metal-oxide particles, salt particles, carbon particles (e.g., one or more allotropes of carbon such as amorphous carbon, graphite, or diamond), or the like. For example, the thermally conductive medium can be a paste of the fatty substance including the solid-particle additive. Advantageously, such a thermally conductive medium can be configured to be compatible with ultrasound imaging, magnetic resonance imaging (“MRI”), computer-assisted tomography (“CT”) scanning, electrocardiogram, or the like.

While not shown, the pad body 148 or 168 can include a film over the patient-interfacing layer 158 of the pad body 148 or the second piece 172 of the pad body 168 individually sealing each well of the wells 178 or each channel of the one-or-more channels 179 in the patient-interfacing side 180 of the patient-interfacing layer 158 or the patient-interfacing side 174 of the second piece 172. The film can be configured for cutting by an instrument (e.g., scalpel), bursting by applied pressure (e.g., squeezing the pad, manipulating the pad over a portion of a patient, etc.), or dissolving upon contact with patient skin or a reagent therefor to expose the thermally conductive medium in the wells 178 or the one-or-more channels 179. In this way, the wells 178 or the one-or-more channels 179 can be advantageously selected as desired by cutting, bursting, or dissolving the film to open the wells 178 or the one-or-more channels 179, thereby by minimizing any mess associated with use of excess thermally conductive medium.

As an alternative to the film over the patient-interfacing layer 158 of the pad body 148 or the second piece 172 of the pad body 168, the pad body 148 or 168 can include a release liner thereover. The release liner can be configured to expose up to an entirety of the patient-interfacing side 180 of the patient-interfacing layer 158 or the patient-interfacing side 174 of the second piece 172 including the thermally conductive medium in the wells 178 or the one-or-more channels 179 when the release liner is removed. That said, in some embodiments, the thermally conductive medium can be separately provided in a same or different package as the one-or-more pads 104. In such embodiments, the release liner can be configured to expose up to an entirety of the patient-interfacing side 180 of the patient-interfacing layer 158 or the patient-interfacing side 174 of the second piece 172 for subsequent addition of the thermally conductive medium to the wells 178 or the one-or-more channels 179 when the release liner is removed. Regardless, the release liner can be beneficial in maintaining an integrity of the patient-interfacing side 180 of the patient-interfacing layer 158 or the patient-interfacing side 174 of the second piece 172.

The pad inlet connector 150 can include a pad inlet configured for charging the conduit layer 154 with the supply fluid, while the pad outlet connector 152 can include a pad outlet configured for discharging the return fluid from the conduit layer 154.

A pad of the one-or-more pads 104 can include a secondary FDL of the one-or-more secondary FDLs 108. For example, the secondary FDL can be pre-connected to the pad as packaged. Alternatively, the secondary FDL can be provided in a same or different package as the pad but not connected to the pad.

The secondary FDL can include secondary tubing 182 configured to convey the supply fluid from the primary FDL 106 when connected to the hydraulic system 112 of the control module 102. Likewise, the secondary tubing 182 can be configured to convey the return fluid back to the primary FDL 106 when connected to the hydraulic system 112 of the control module 102.

The secondary FDL can be split at a pad-connecting end of the secondary FDL. The pad-connecting end of the secondary FDL can include a pair of pad-connecting secondary FDL connectors. A pad-connecting secondary FDL outlet connector 184 of the pair of pad-connecting secondary FDL connectors can be configured to fluidly connect to the pad inlet connector 150. A pad-connecting secondary FDL inlet connector 186 of the pair of pad-connecting secondary FDL connectors can be configured to fluidly connect to the pad outlet connector 152.

The secondary FDL need not be split at a primary FDL-connecting end of the secondary FDL like the pad-connecting end of the secondary FDL. Indeed, an unsplit primary FDL-connecting end of the secondary FDL facilitates quickly connecting the one-or-more secondary FDLs 108 to the primary FDL 106. Accordingly, the primary FDL-connecting end of the secondary FDL can include a single primary FDL-connecting secondary FDL connector 188 configured to fluidly connect to the secondary FDL-connecting primary FDL connector (not shown) set forth above. The primary FDL-connecting secondary FDL connector 188 can include both an inlet and an outlet corresponding to the outlet and the inlet of the secondary FDL-connecting primary FDL connector.

### Packages for TTM

While not shown, a package for TTM can include the one-or-more pads 104, the one-or-more secondary FDLs 108 corresponding in number to the one-or-more pads 104, instructions for use, and packaging around at least the one-or-more pads 104, the one-or-more secondary FDLs 108, and the applicator as the instructions for use can be printed on the packaging. The one-or-more pads 104 can include the one-or-more secondary FDLs 108 respectively pre-connected thereto in the pad package. Alternatively, the one-or-more pads 104 are not connected to the one-or-more secondary FDLs 108 in the package. In other embodiments, the one-or-more pads 104 and the one-or-more secondary FDLs 108 can even be provided in different packages.

If the one-or-more pads 104 include the wells 178 or the one-or-more channels 179 in the patient-interfacing side 180 of the patient-interfacing layer 158 of the pad body 148 or the patient-interfacing side 174 of the second piece 172 of the pad body 168 without the thermally conductive medium disposed therein, the package can further include a container or applicator including the thermally conductive medium for addition to the wells 178 or the one-or-more channels 179.

### Methods

Methods of the system 100 or the one-or-more pads 104 include methods of use. For example, a method of using the system 100 for TTM can include one or more steps selected from a thermally conductive medium-readying step, a pad-placing step, an FDL-connecting step, a pad-charging step, and a fluid-circulating step.

The pad-placing step can include placing at least a portion of a pad of the one-or-more pads 104 on a patient's body in contact with skin S of the patient's body. (See, for example, FIG. 3A.) Depending upon the pad of the one-or-more pads 104 set forth above, the portion of the pad placed in contact with the skin of the patient's body can include the wells 178 or the one-or-more channels 179 in the patient-interfacing side 180 of the patient-interfacing layer 158 of the pad body 148 or the patient-interfacing side 174 of the second piece 172 of the pad body 168, which wells 178 or one-or-more channels 179, in turn, include the thermally conductive medium disposed therein. Alternatively, the portion of the pad placed in contact with the skin of the patient's body can include the hydrogel on the patient-interfacing side 174 of the second piece 172 of the pad body 168 instead of the wells 178, the one-or-more channels 179, or the thermally conductive medium therein.

The pad-placing step can further include placing the first piece 170 of the pad over the second piece 172 of the pad for embodiments of the pad including the two-piece pad body 168.

The thermally conductive medium-readying step can be performed before the pad-placing step or together with the pad-placing step depending upon the pad of the one-or-more pads 104 set forth above.

As set forth above, the pad body 148 or 168 can include the film over the patient-interfacing layer 158 of the pad body 148 or the second piece 172 of the pad body 168 individually sealing each well of the wells 178 or each channel of the one-or-more channels 179 in the patient-interfacing side 180 of the patient-interfacing layer 158 or the patient-interfacing side 174 of the second piece 172. In such embodiments, the thermally conductive medium-readying step can include a film-cutting step, a film-bursting step, or a film-dissolving step. The film-cutting step can include cutting the film over the wells 178 or the one-or-more channels 179 with an instrument (e.g., scalpel) to expose the thermally conductive medium in any one or more individually sealed wells of the wells 178 or channels of the one-or-more channels 179. The film-bursting step can include bursting the film over the wells 178 or the one-or-more channels 179 with applied pressure (e.g., squeezing the pad, manipulating the pad over the patient, etc.) to expose the thermally conductive medium in any one or more individually sealed wells of the wells 178 or channels of the one-or-more channels 179. The film-dissolving step can include dissolving the film over the wells 178 or the one-or-more channels 179 upon contact with the patient's skin or an applied reagent to expose the thermally conductive medium in any one or more individually sealed wells of the wells 178 or channels of the one-or-more-channels 179.

As set forth above, the pad body 148 or 168 can include the release liner over the patient-interfacing layer 158 of the pad body 148 or the second piece 172 of the pad body 168 as an alternative to the film. In such embodiments, the thermally conductive medium-readying step can include a release liner-removing step. The release liner-removing step can include removing the release liner from the pad to expose the wells 178 or the one-or-more channels 179 including the thermally conductive medium therein. However, in some embodiments set forth above, the thermally conductive medium can be separately provided in a container or applicator in a same or different package as the pad. In such embodiments, the release liner-removing step can be combined with a dispensing step. Indeed, subsequent to performing the release liner-removing step, which, in this case, exposes the wells 178 or the one-or-more channels 179 without the thermally conductive medium disposed therein, the dispensing step can be performed. Such a dispensing step can include dispensing the thermally conductive medium into the wells 178, into the one-or-more channels 179, or onto the patient. That said, if the thermally conductive medium is dispense on the patient the release liner-removing step can be performed thereafter if desired.

Notwithstanding the foregoing, the release liner can alternatively be over the hydrogel on the patient-interfacing side 174 of the second piece 172 of the pad body 168 instead of the wells 178 or the one-or-more channels 179. In such embodiments, the release liner-removing step can include removing the release liner from the patient-facing side 174 of the second piece 172 of the pad body 168 to expose the hydrogel.

The method can further include another release liner-removing step for embodiments of the pad including the two-piece pad body 168. Indeed, the other release liner-removing step can include removing the release liner from the pad-facing side 176 of the second piece 172 of the pad body 168 to expose the adhesive thereon for adhering the first and second pieces 170 and 172 of the pad body 168 together upon placing the first piece 170 over the second piece 172 in the pad-placing step. However, if the first piece 170 of the pad body 168 includes the adhesive instead of the second piece 172 of the pad body 168, the release liner-removing step can include removing the release liner from the impermeable film 156 of the first piece 170 to expose the adhesive thereon for adhering the first and second pieces 170 and 172 of the pad body 168 together upon placing the first piece 170 over the second piece 172 in the pad-placing step.

If a secondary FDL is not pre-connected to the pad, the method can include the FDL-connecting step. The FDL-connecting step can include fluidly connecting the pad-connecting secondary FDL outlet connector 184 at the split pad-connecting end of the secondary FDL to the pad inlet connector 150. The FDL-connecting step can also include fluidly connecting the pad-connecting secondary FDL inlet connector 186 at the split pad-connecting end of the secondary FDL to the pad outlet connector 152.

The pad-charging step can include charging the one-or-more conduits 164 of the conduit layer 154 of the pad body 148 or 168 with the supply fluid of the temperature-controlled fluid. As set forth above, the supply fluid can be provided by the hydraulic system 112 of the control module 102 by way of a combination of fluidly connected FDLs including the secondary FDL and the primary FDL 106.

The fluid-circulating step can include circulating the supply fluid through the conduit layer 154 of the pad body 148 or 168 to cool or warm the patient's body as needed in accordance with TTM. Indeed, the fluid-circulating step can include transferring heat between the temperature-controlled fluid and the patient's body by thermal conduction through the conduit layer 154. For example, the fluid-circulating step can include circulating a cool fluid through the conduit layer 154 to bring the patient into hypothermia from normothermia. In another example, the fluid-circulating step can include circulating a warm fluid through the conduit layer 154 to bring the patient into normothermia from hypothermia. In yet another example, the fluid-circulating step can include circulating a warm fluid through the conduit layer 154 to bring the patient into hyperthermia from normothermia. In yet another example, the fluid-circulating step can include circulating a cool fluid through the conduit layer 154 to bring the patient into normothermia from hyperthermia.

While the method set forth above is described with reference to a single pad of the one-or-more pads 104, it should be understood that any number of pads of the one-or-more pads 104 can be used as necessary to effectuate a desired treatment by way of the system 100 for TTM.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A pad for targeted temperature management (“TTM”), comprising:
a multilayered pad body including:
a conduit layer including one or more conduits configured to convey a temperature-controlled fluid as a supply fluid from a hydraulic system and convey a return fluid back to the hydraulic system; and
a patient-interfacing layer over the conduit layer configured for placement on a portion of a patient's body, the patient-interfacing layer having a patient-facing side including a plurality of wells configured to hold a thermally conductive medium;
a pad inlet connector including a pad inlet configured for charging the conduit layer with the supply fluid; and
a pad outlet connector including a pad outlet configured for discharging the return fluid from the conduit layer.

2. The pad of claim 1, wherein the wells are square-packed rectangular wells.

3. The pad of claim 1, wherein the wells are hexagonally packed circular wells.

4. The pad of claim 1, wherein the wells are hexagonally packed hexagonal wells.

5. The pad of claim 1, wherein the wells are interconnected.

6. The pad of claim 1, wherein the pad includes the thermally conductive medium in the wells.

7. The pad of claim 6, wherein the thermally conductive medium includes a polymer, a gel, or a fatty substance.

8. The pad of claim 7, wherein the thermally conductive medium includes a solid-particle additive including metal flakes, metal-oxide particles, salt particles, or carbon particles.

9. The pad of claim 8, wherein the thermally conductive medium is a paste of the fatty substance including the solid-particle additive.

10. The pad of claim 6, the pad body further including a film over the patient-interfacing layer individually sealing each well of the plurality of wells, the film configured for cutting by an instrument, bursting by applied pressure, or dissolving upon contact with skin or a reagent to expose the thermally conductive medium in the wells.

11. The pad of claim 6, further comprising a release liner over the patient-interfacing layer, the release liner configured to expose the thermally conductive medium in the wells when the release liner is removed.

12. The pad of claim 1, further comprising a release liner over the patient-interfacing layer, the release liner configured to expose the wells when the release liner is removed for subsequent addition of the thermally conductive medium to the wells.

13. The pad of claim 1, the pad body further including an impermeable film between the patient-interfacing layer and the conduit layer configured to retain the temperature-controlled fluid in the conduit layer.

14. The pad of claim 13, wherein the conduit layer includes a plurality of protrusions extending from the conduit layer toward the impermeable film, the protrusions configured to promote even flow of the temperature-controlled fluid.

15. The pad of claim 1, further comprising a secondary fluid delivery line (“FDL”) configured to convey the supply fluid from the hydraulic system and convey the return fluid back to the hydraulic system, the secondary FDL split at a pad-connecting end of the secondary FDL, and the pad-connecting end of the secondary FDL including a pair of secondary FDL connectors including a secondary FDL outlet connector configured to fluidly connect to the pad inlet connector and a secondary FDL inlet connector configured to fluidly connect to the pad outlet connector.

16. A pad package for targeted temperature management (“TTM”), comprising:
one or more pads for TTM, each pad of the one-or-more pads including:
a multilayered pad body including:
a conduit layer including one or more conduits configured to convey a temperature-controlled fluid as a supply fluid from a hydraulic system and convey a return fluid back to the hydraulic system; and
a patient-interfacing layer over the conduit layer configured for placement on a portion of a patient's body, the patient-interfacing layer having a patient-facing side including a plurality of wells configured to hold a thermally conductive medium;
an applicator including the thermally conductive medium for addition of the thermally conductive medium to the wells; and
packaging around the one-or-more pads and the applicator.

17. The pad package of claim 16, wherein the wells are square-packed rectangular wells, hexagonally packed circular wells, or hexagonally packed hexagonal wells.

18. The pad package of claim 16, wherein the wells are interconnected.

19. The pad package of claim 16, wherein the thermally conductive medium includes a polymer, a gel, or a fatty substance.

20. The pad package of claim 16, wherein the thermally conductive medium includes a solid-particle additive including metal flakes, metal-oxide particles, salt particles, or carbon particles.

21. The pad package of claim 16, each pad of the one-or-more pads further comprising a release liner over the patient-interfacing layer, the release liner configured to expose the wells when the release liner is removed for subsequent addition of the thermally conductive medium to the wells.

22. The pad package of claim 16, the pad body further including an impermeable film between the patient-interfacing layer and the conduit layer configured to retain the temperature-controlled fluid in the conduit layer.

23. The pad package of claim 16, each pad of the one-or-more pads further including:
a pad inlet connector including a pad inlet configured for charging the conduit layer with the supply fluid; and
a pad outlet connector including a pad outlet configured for discharging the return fluid from the conduit layer.

24. The pad package of claim 23, further comprising one or more secondary fluid delivery lines (“FDLs”) corresponding in number to the one-or-more pads, each secondary FDL of the one-or-more secondary FDLs configured to convey the supply fluid from the hydraulic system and convey the return fluid back to the hydraulic system, each secondary FDL of the one-or-more secondary FDLs split at a pad-connecting end of the secondary FDL, and the pad-connecting end of the secondary FDL including a pair of secondary FDL connectors including a secondary FDL outlet connector configured to fluidly connect to the pad inlet connector and a secondary FDL inlet connector configured to fluidly connect to the pad outlet connector.

25-75. (canceled)
